(19) United States
(12) Patent Application Publication
(10) Pub. No.: US 2011/0040170 A1
(43) Pub. Date: Feb. 17, 2011

(54) ENDOSCOPIC IMAGING PHOTODYNAMIC THERAPY SYSTEM AND METHODS OF USE

(75) Inventors: Eli Geva, Ramat HaSharon (IL); Yoram Salomon, Rehovot (IL); Avigdor Scherz, Newe Amit (IL); Itai Glinert, Kfar-Saba (IL)

Correspondence Address: Fleit Gibbons Gutman Bongini & Bianco PL, 21355 EAST DIXIE HIGHWAY, SUITE 115, MIAMI, FL, 33180, US

(73) Assignee: YEDA RESEARCH AND DEVELOPMENT CO., LTD., Rehovot (IL)

(21) Appl. No.: 12/865,050

(22) PCT Filed: Jan. 28, 2009
(86) PCT No.: PCT/IL09/00108

§ 371 Date: Oct. 5, 2010

Related U.S. Application Data
(60) Provisional application No. 61/023,912, filed on Jan. 28, 2008.

(51) Int. Cl. A61N 5/00; A61B 1/00; A61B 8/12; A61B 5/055

(52) U.S. Cl. 600/411; 604/20; 604/21; 600/104; 600/439

(57) ABSTRACT

The invention provides an endoscopic imaging photodynamic therapy system (EIPS) for focused tissue ablation by illumination of a photosensitizer drug in a target tissue, said system comprising an endoscopic assembly, a real-time imaging component for locating the target tissue and monitoring the ablation intervention, a therapeutic light system and, optionally, a drug delivery module, wherein said imaging component comprises a flexible transducer with an operative channel for insertion of a flexible light guide of the therapeutic light system and, optionally, a flexible drug delivery catheter of the drug delivery module. This EIPS may be used in various medical applications where tissue ablation is required and photodynamic therapy may be applied, in particular, in the treatment of extrauterine pregnancy (EUP).

### TECHNICAL FIELD

The present invention relates to an endoscopic imaging photodynamic therapy system for focused tissue ablation and methods of use.

### BACKGROUND ART

### Tissue Ablation

Ablation, as used in Medicine, is defined as removal or excision of a body part or tissue or its function and is usually carried out surgically. Ablation may also be performed by the administration of hormones, drugs, radiofrequency, heating, freezing and/or any other suitable method for performing ablation. For example, surface ablation in the skin can be carried out by chemicals (peeling) or by lasers in order to remove skin spots, aged skin or wrinkles, and in otolaringology for several kinds of surgery, such as prevention of snoring. Surface ablation of the cornea for several types of eye refractive surgery is now common, using laser ablation, for example, to remodel the cornea refractive properties in order to correct refraction errors, such as astigmatism, myopia and hyperopia.

Radiofrequency ablation (RFA) is the most popular minimally invasive thermal ablation technique worldwide. RFA employs radiofrequency energy to destroy abnormal electrical pathways in heart tissue and is used, for example, to cure a variety of arrhythmias such as supraventricular tachycardia, WPW syndrome, ventricular tachycardia and atrial fibrillation. The energy emitting probe (electrode) is placed into the heart through a catheter. New ablation techniques include cryoablation, microwave ablation, and high intensity focused ultrasound (HIFU) ablation, in which acoustic energy is used.

RFA expanded the treatment options for certain oncology patients. Minimally invasive, image-guided therapy may now provide effective local treatment of isolated or localized neoplastic disease, and can also be used as an adjunct to conventional surgery, systemic chemotherapy, or radiation. Other clinical applications of RFA include treatment of patients with liver cancers, kidney, adrenal, and prostate tumors; benign prostatic hyperplasia; painful or abnormal neural tissue; and painful soft tissue or bone masses that are unresponsive to conventional therapy.

### Photodynamic Therapy (PDT)

Photodynamic therapy (PDT) is a relatively new treatment modality best known for its applications in the therapy of cancer and macular degeneration. PDT is rapidly maturing in the clinic with the development of new photosensitizers, treatment protocols and additional clinical applications as well as increasing basic understanding of this technique. In the US, several FDA approved PDT drugs are in use and others are in various stages of preclinical and clinical trials.

PDT involves two non-toxic components that are combined at the treatment site to induce cellular and tissue damage in an oxygen-dependent manner: a non-toxic photosensitizer drug, administered systemically or locally, and non-hazardous light of a matched wavelength that is delivered locally to the treatment site. The photosensitization of the drug elicits the transfer of energy or an electron to molecular oxygen resulting in instant local generation of cytotoxic reactive oxygen species (ROS). Depending on the drug and the treatment protocol, phototoxicity can be directed toward the targeted tissue or tumor cells or towards the respective vasculature. The half-life of these radicals in the biological milieu is extremely short (<0.04 μs) restricting their diffusion distance to <0.02 μm, practically confining the damage to the illuminated area. Compared to surgical resection of tumors, PDT following I.V. administration of the photosensitizer can be delivered to internal lesions via optic fibers. Thus, PDT can be defined as a highly controlled, minimally-invasive, local treatment. In contrast to other clinical laser-ablation techniques, in PDT low energy lasers are commonly used, which deliver a few hundred mW/treatment site.

Devices and methods for photodynamic ablation of tissues have been described. U.S. Pat. No. 6,811,562 discloses procedures and devices for photodynamic cardiac ablation therapy for treating cardiac tissue by forming lesions in that tissue using said PDT techniques. WO 97/06797 discloses PDT using green porphyrins such as BPD for endometrial ablation to treat endometrial disorders such as dysfunctional uterine bleeding, menorrhagia, endometriosis, endometrial neoplasia, sterilization and termination of early pregnancy. No device is disclosed.

Extrauterine pregnancy (EUP)

Extrauterine pregnancy (EUP) in humans is the abnormal implantation of an embryo outside the uterus. The prevalence of EUP is about 10-20 cases per 1000 pregnancies. During the 1980's and 1990's there has been a 3-4 fold increase in EUP incidence in developed countries due to increase in the use of assisted reproductive technology and prevalence of pelvic inflammatory disease. Other risk factors include infertility, previous EUP and pelvic surgery. The high occurrence rate of EUP makes it the second leading cause of overall pregnancy-related maternal mortality in the USA and the leading cause of pregnancy-related maternal death during the first trimester.

Early diagnosis is the key to successful treatment of EUP. Intervention prior to Fallopian tube rupture allows conservative treatment and enhances fertility preservation. Today most cases are diagnosed early in the first trimester of pregnancy by a combination of transvaginal ultrasonography and determination of serum β-human chorionic gonadotropin (β-hCG) levels.

Current treatment options for EUP consist of medical or surgical therapy. Medical therapy with methotrexate is aimed against the rapidly dividing cells of the placenta and embryo. Methotrexate, a chemotherapeutic drug, is a powerful anti-metabolite that inhibits dihydrofolate reductase, inhibiting DNA replication and cell division. The adverse effects of methotrexate include acute abdominal pains, impaired liver function, stomatitis, cytopenia and rarely, pneumonitis. However, medical therapy is an established treatment of EUP only in selected patients (e.g., embryonic mass size of less than 4 cm, absence of fetal heart beat and low blood β-hCG levels), with a success rate of 70-95%.

A large proportion of patients with EUP will require surgical treatment, either conservative (salpingostomy) or radical (salpingectomy). Conservative surgery aims at preserving the Fallopian tube and consequent fertility by removing only the implanted embryo and placenta. The main risk factor associated with this technique is incomplete removal of the placenta, which can result in persistent disease, necessitating further surgery or methotrexate treatment and constituting treatment failure (˜15% of patients). Radical surgery involves the resection of the Fallopian tube with the pregnancy, ending the medical emergency with high certainty, but usually resulting in impaired fertility. In addition, surgery entails other risks such as infection, hemorrhage and anesthesia, as well as a risk for pelvic adhesions and mechanical infertility. Prolonged hospitalization and recovery times make surgery significantly more costly when compared to medical treatment.

The high prevalence of EUP, as well as the drawbacks and limitations of current treatment options, prompt a search for novel treatment modalities.

The similarities between tumors and newly implanted pregnancies are striking: both develop on the basis of a rapidly dividing cell mass that invades surrounding tissues and induce angiogenesis by establishing a neo-vascular system. In spite of this similarity, a single study attempting photo-ablation of EUP was not successful (Yang et al., 1993). In this study, Yang et al. attempted photo-ablation of EUP in the pregnant rat using systemic administration of 5-aminolevulinic acid (5-ALA) combined with illumination of an entire uterine horn. This resulted in the termination of all pregnancies in the treated horn, as well as subsequent high infertility rates (only 66.2% of treated animals developed pregnancies in the treated horn, presenting ˜28% fewer implanted embryos) indicative of lasting endometrial damage. A subsequent study by the same group reported the non-selective ablation of all the embryos in a rat uterine horn following systemic injection of 5-ALA and illumination (Yang et al., 1994). Although reviewed as recently as 2000 by the same group (Reid et al, 2000), no follow up in the direction of EUP ablation has been published, but rather the group's attention has shifted to endometrial ablation as a potential treatment for endometriosis by 5-ALA PDT (Yang et al., 1996; Krzemien, 2002).

### SUMMARY OF INVENTION

The present invention is directed toward a novel technological platform designed for optimal delivery of minimally invasive internal treatments by photodynamic means under controlled real-time imaging.

In one aspect, the present invention relates to an endoscopic imaging photodynamic therapy system for focused tissue ablation by illumination of a photosensitizer drug in a target tissue, said system comprising an endoscopic assembly, a real-time imaging component for locating the target tissue and monitoring the ablation intervention, a therapeutic light system and, optionally, a drug delivery module, wherein said imaging component comprises a flexible transducer with an operative channel for insertion of a flexible light guide of the therapeutic light system and, optionally, a flexible drug delivery catheter of the drug delivery module.

In another aspect, the invention provides a method for focused tissue ablation in a target tissue of an individual in need using the endoscopic imaging photodynamic therapy system of the invention.

The system and method of the present invention can be used for treatment of various diseases, disorders and conditions by focused tissue ablation and, particularly, for photodynamic ablation of the fetoplacental unit(s) in the treatment of extrauterine pregnancy (EUP).

### BRIEF DESCRIPTION OF DRAWINGS

In the accompanying drawings, like reference characters relate to similar features in the different views to facilitate comparison.

FIGS. 1A-1E are schematic illustrations of one embodiment of the endoscopic imaging photodynamic therapy system (EIPS) of the invention and components thereof: 1A—Endoscopic assembly; 1B—Real-time imaging component; 1C—Drug delivery module; 1D—Therapeutic light system; 1E—Flexible service catheter.

FIGS. 2A-2E depict different presentations of the connection between the flexible transducer of the real-time imaging component and the operative channel.

FIGS. 3A-3B are schematic illustrations of intrauterine insertion of the EIPS of FIG. 1 designed for reproductive tract intervention showing the feto-placental unit in the Fallopian tube in a case of extrauterine pregnancy (EUP).

FIGS. 4A-4D depict flexible transducer (4A) and needle (4B) insertion, drug injection (4C), optic fiber insertion and therapeutic illumination (4D) in the EUP model, respectively.

FIGS. 5A-5C depict PMRDA-uterine PDT experimental layout and results. (5A) The layout of the rat placental PDT procedure is presented during the illumination step (for details see Material and Methods) (5B). Exposed rat uteri with embryos selected for treatment (marked by yellow circles) at PDT day (E14, upper left panel) or 48 h after PMRDA-PDT (E16, lower left panel) or LC/DC controls before (E14, upper right panel) or 48 h after treatment (E16, lower right panel) are presented. Macroscopic in utero analysis of PDT-induced damage to the selected feto-placental unit (shrinkage and discoloration, lower left panel) and unharmed embryos following control manipulation (normal size and color, lower right panel) can be observed. (5C) Uterine PMRDA-PDT summary of results: bars represent embryo-placental unit destruction as embryo death rates, following PMRDA-PDT (11/14 embryos, 78.6%), LC (1/8 embryos, 12.5%) and DC (3/8 embryos, 37.5%). Dashed line represents death rate of untreated embryos (UN, 13/230 embryos, 5.7%) in treated rats. PMRDA is palladium 3¹-oxo-15-methoxy-carbonylmethylrhodobacteriochlorin-13¹,17³-di(2-N²-dimethylamino ethyl) amide. E14 and E16 are embryonic days 14 and 16, respectively. LC is light control. DC is dark control, as described in “In vivo PDT protocol”, in Materials and Methods.

FIGS. 6A-6J depict histological presentation of utero-placental tissues in untreated placentas (E16) (6A-6F) and following PMRDA-PDT (6G-6J): (6A) Overview of intact placenta at E16. (6B) Heavily vascularized uterine wall with blood vessel (Bv.). (6C) Labyrinth layer. (6D) Spongiotrophoblast layer. (6E) Overview of intact embryo. (6F) Magnification of well-defined, intact structures (Vt.—vertebra, Ln.—lung, Ht.—heart, Lv.—liver). (6G) Overview of PMRDA-PDT treated placenta and embryo at E16 (Ut.—uterus). (6H) Partially dissolved, heavily necrotic embryo, containing ill-defined structures (Vt.—vertebra). (6I) Damaged placenta with immune-cell-infiltrate (Nif.) and visible hemorrhage (Hm.). (6J) Damaged placental blood vessel (Bv.). Scale bars: in 6A, 6E and 6G, 1 mm, in 6B-6D, 6F and 6H-6J, 100 μm.

FIGS. 7A-7C depict fertility assessment in post PDT rats. (7A) A rat uterus from a gestating rat (˜E8), in its second pregnancy (following PDT, parturition and subsequent mating) was examined to verify implantation in both uterine horns. Em.—embryonic sac. Cv.—cervix. Implanted embryonic sacs are evident in both uterine horns. (7B) MRI of uterus in a similarly treated rat (˜E16). Circles mark embryonic sacs in utero, and arrow marks cervix. Implantation is evident in both uterine horns. (7C) Post partum litter of PDT treated rat (imaged in 7B), showing normal, healthy pups.

FIGS. 8A-8I depict histolopathological analysis of uteri of PMRDA-PDT rats following parturition and pup weaning. (8A) Post PDT uterus sampled ˜22d after parturition (right horn—untreated, left horn—PDT). The uterine horns were separated, fixed in carnoy's fixative and embedded in paraffin, and sections were then prepared from the untreated- and the post PDT-uterine horn ((8B-E and 8F-I, respectively) and stained as follows: H&E (8B and 8F), anti-SMA antibody (8C and 8G)—showing smooth muscle layer of uterine wall, anti-pan-cytokeratin antibody (8D and 8H)—showing uterine endometrium layer, and anti-vWF antibody (8E and 8I)—showing uterine vasculature. Histological analysis shows no pathological findings in either uterine horn (post PDT or untreated), both presenting minimal, within normal limits, lesions and without any necrotic regions. Scale bars: 8A—1 cm, 8B-8D, 8F-8H—200 μm and 8E and 8I—100 μm.

### MODES FOR CARRYING OUT THE INVENTION

The present invention provides a technological platform based on an endoscopic imaging photodynamic therapy system (EIPS) designed for optimal delivery of minimally invasive internal treatments by photodynamic means under controlled real-time imaging, in which the photosensitizer administration can be done locally or systemically.

The EIPS of the present invention generally consists of three major components that act in concert to provide the tasks needed to perform the procedure accurately and safely while the various instruments, control panels and monitor(s) are placed at the patient's bedside, conveniently situated for controlled operation by the physician.

The EIPS of the invention is suitable for transvaginal focused tissue ablation, particularly for ablation of the feto-placental unit in ectopic location in cases of extrauterine pregnancy (EUP). Such a system is described hereinbelow wherein the intravaginally inserted assembly contains the respective front-end components for controlled interactive function at the treatment site. However, with appropriate modifications, the EIPS can also become instrumental in other medical applications where PDT may be applied for treatment such as, but not limited to, malignant and pre-malignant lesions, gynecological diseases, cardiology and other cardiovascular diseases, gastrointestinal tract lesions, respiratory system diseases, urinary tract diseases, musculo-skeletal diseases, head and neck or neuronal and brain treatments.

In one aspect of the invention, an endoscopic imaging photodynamic therapy system is provided for focused tissue ablation by illumination of a photosensitizer drug in a target tissue, said system comprising an endoscopic assembly, a real-time imaging component for locating the target tissue and monitoring the ablation intervention, a therapeutic light system and, optionally, a drug delivery module, wherein said imaging component comprises a flexible transducer with an operative channel for insertion of a flexible light guide of the therapeutic light system and, optionally, a flexible drug delivery catheter of the drug delivery module.

As defined herein, the term “target tissue” refers to any biological tissue or a part thereof, including blood and/or lymph vessels, which is the object of focused tissue ablation and includes, for example, a group of cells, a tissue, a body part or an organ. The target tissue may also be an embryo/fetus or a placenta or part thereof when EUP is treated.

In one embodiment, the present invention provides an EIPS wherein:

(a) said endoscopic assembly comprises a control handle, an operation handle and an application adaptor;

(b) said real-time imaging component comprises means for guidance for location of said target tissue and monitoring of the ablation intervention in said target tissue, and a flexible transducer with an operative channel;

(c) said therapeutic light system consists of a light source, a flexible light guide and an operating switch for the light system; and

(d) said drug delivery module, if present, comprises a flexible drug delivery catheter adapted for injecting a photosensitizer drug to the target tissue, a drug delivery means and a photosensitizer drug in an injectible form.

According to one embodiment of the invention, the flexible light guide of the therapeutic light system and the flexible drug delivery catheter of the drug delivery module, if present, are inserted into the operative channel of the flexible transducer of the real-time imaging component, for example, via a flexible service catheter.

The control handle of the endoscopic assembly (a) may be manual or computer-controlled and comprises a proximal grip and at least one service opening. When there are two service openings, one is used for insertion of the flexible transducer of the real-time imaging component, as described below, and the other may be used, for example, for washing the tissue, suction from the tissue, insertion of needle biopsies to sample cells from an abnormal area for laboratory testing, removal of a piece of a polyp, a gallstone, a foreign object, or a stent, etc.

The operation handle of the endoscopic assembly preferably comprises means for aiming and bending the flexible transducer with the flexible drug delivery catheter, if present, and the flexible light guide towards the target tissue. Said means may be mechanical such as a navigator dial or computer-aided, computer-controlled, computer-operated or wireless.

The real-time imaging component for locating the target tissue and monitoring the ablation intervention may be any imaging component such as, without limitation, ultrasound (US), magnetic resonance imaging (MRI), computerized tomography (CT), positron emission tomography (PET), light-based video, or any combination thereof, or any other or future imaging technique, and may also be used to measure the size of the target tissue, when appropriate.

Any appropriate light source may be used in the therapeutic light system such as a diode laser, preferably with several variable output channels. Preferably, the diode laser emits a light beam with a wavelength that matches one or more of the absorption peaks of the photosensitizer drug. The operating switch for the therapeutic light system may be a pedal.

In one embodiment of the invention, the flexible light guide of the therapeutic light system is equipped with front-end optics to improve viewing and location of the target tissue.

According to one embodiment of the invention, the flexible light guide of the therapeutic light system is inserted into the target tissue or to its close proximity simultaneously with the flexible drug delivery catheter of the drug delivery module via the operative channel of the flexible transducer. In another embodiment, the flexible light guide is inserted into the target tissue or to its close proximity following the insertion of the flexible drug delivery catheter, which needs retraction of the flexible drug delivery catheter prior to insertion of the flexible light guide.

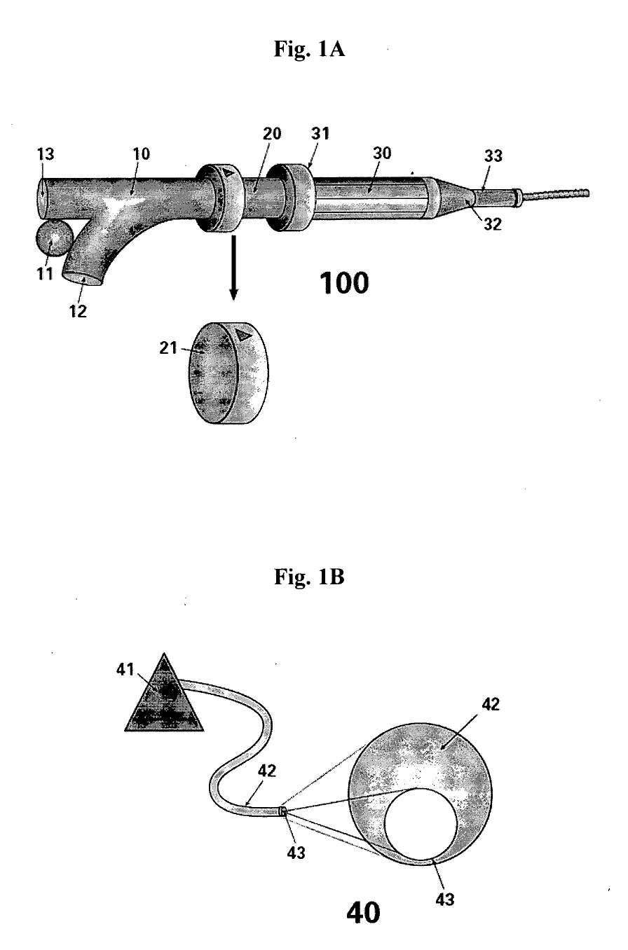

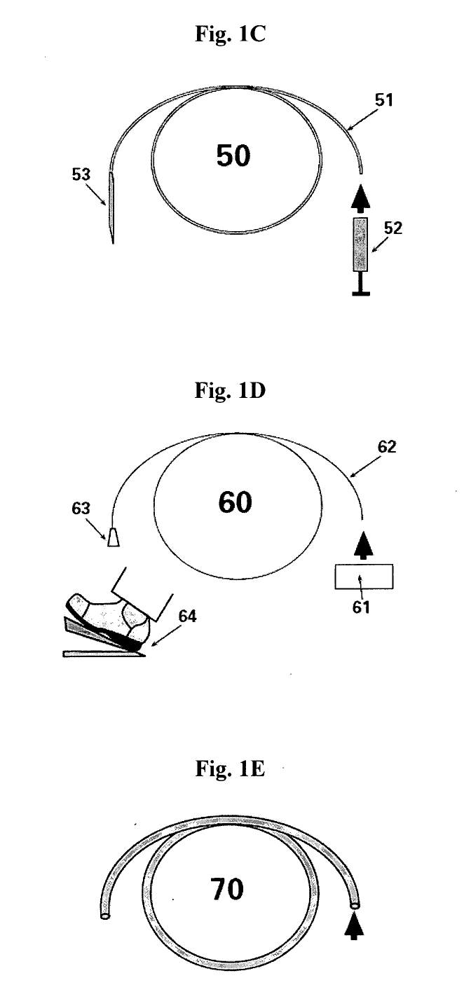

FIG. 1 depicts one embodiment of the EIPS of the invention comprising: an endoscopic assembly 100 (1A); a real-time imaging component 40 (1B); a drug delivery module 50 (1C); a therapeutic light system 60 (1D); and a flexible service catheter 70 (1E).

FIG. 1A schematically illustrates one embodiment of the endoscopic assembly 100 of the invention, comprising a control handle 10, an operation handle 20 and an application adaptor 30. In this embodiment, the control handle 10 is designed for manual control and comprises a proximal grip 11, a lower service opening 12 and an upper service opening 13. However, the control handle may also be designed as a computer-aided or computer-controlled handle, e.g., joystick, mouse or else.

The service openings may also be in different positions depending on the engineering of the device, for example, one to the left and the other to the right. One of the service openings is used for insertion of the flexible transducer of the real-time imaging component while the other service opening may be used for different purposes as described above.

The operation handle 20 of the endoscopic assembly 100 may comprise a navigator dial 21 that allows aiming and bending of the flexible service catheter 70 and the real-time imaging component 40 towards the target tissue. The application adaptor 30 described is designed for reproductive tract intervention and is connected to the operation handle 20 through a connecting screw 31 and to a flexible guide 33 through the non-flexible guide 32. However, the size and shape of the application adaptor 30 may be modified according to the specific use and the target tissue and location involved in the procedure.

FIG. 1B illustrates an embodiment of the real-time imaging component 40 of the invention comprising imaging system 41 and means for guidance for location of the target tissue and monitoring of the ablation intervention in the target tissue (including spatial orientation and blood flow), consisting of a flexible transducer 42 that includes the operative channel 43 through which the flexible service catheter 70 (see FIG. 1E) is inserted. In one embodiment, when the intervention is transvaginal, the vaginally inserted flexible transducer 42 and the flexible service catheter 70 are appropriately presented for optimal relay of the streaming image to the external monitor of the imaging component 40.

According to the present invention, the real-time imaging component 40 for locating the target tissue and monitoring the ablation intervention, may be an ultrasound (US), magnetic resonance imaging (MRI), computerized tomography (CT), positron emission tomography (PET), light-based video, and any other known imaging component or developed in the future that is suitable for imaging biological material, or any combination thereof In one preferred embodiment, the real-time imaging component is Doppler ultrasound (US Doppler) that can detect and measure vascular blood flow. The real-time imaging component 40 may also be used for measuring the size of the target tissue, for example, the size of the embryo when the procedure is used for ablation/treatment of extrauterine pregnancy.

FIG. 1C illustrates an embodiment of a drug delivery module 50 of the invention comprising a disposable flexible drug delivery catheter 51, a drug delivery means 52, a needle 53 and a therapeutic photosensitizer drug in an injectible form. In the embodiment illustrated herein, the drug delivery module is adapted for injecting the photosensitizer drug to the target tissue and the drug delivery means 52 is illustrated as a syringe, but any other means suitable for injection of a drug is encompassed by the invention. While the photosensitizer drug solution to be injected is contained within the drug delivery means 52, the needle 53 is positioned at the tip of the flexible drug delivery catheter ready for optimal injection into the tissue. The photosensitizer may be contained in a capsule that may be of various volumes to accommodate photosensitizer doses adapted to the target tissue character (tissue type, size, etc.). The photosensitizer drug is released from the capsule upon operation of the syringe 52 or any other drug delivery means that can control the delivery of the drug at the external service end of the endoscopic assembly. Said capsule may be included in the drug delivery means 52, flexible drug delivery catheter 51 or the needle 53.

The photosensitizer drug used in the invention may be any known photosensitizer as well as any photosensitizer to be developed that is suitable for PDT, and is chosen according to the target tissue to be treated. The photosensitizer drug may be systemically injected to the patient, in which case the drug delivery module function may be either absent in the system or it is present in the system, but is not used in the intervention.

FIG. 1D illustrates an embodiment of a therapeutic light system 60 of the invention consisting of a light source 61, a flexible light guide 62 with an appropriate optic lens/diffuser 63 and an operating switch 64.

The light source 61 may be any suitable light source and is preferably a diode laser that emits a light beam with a wavelength that matches one of the absorption peaks of the photosensitizer drug, preferably a diode laser with several variable output channels. In a specific embodiment, the diode laser is a standard PDT diode-laser (optionally with several 0.05-4W variable output channels) as used, for instance, in clinical PDT of prostate cancer treatment, situated at the patient bedside. The therapeutic light dose is delivered via one or more flexible light guides 62 equipped with optional front-end optics (diffuser or lens 63 for interstitial light delivery), integrated in the light system for optimal function and represented at the assembly tip for optimal insertion and delivery of the therapeutic light dose. In an internal body space like bladder (e.g., inflated bladder), non-interstitial illumination is provided using a light beam originating from the optic fiber tip. When the procedure is transvaginal for treatment of EUP, the flexible light guide 62 is best situated to guide the intervention towards the target tissue to be ablated, located for example, in the Fallopian tube, uterine isthmus or cervix for maximal efficacy and safety of the treatment. The therapeutic illumination time is estimated to be a matter of minutes.

The wavelength of the laser is matched with one of the absorption peaks of the selected photosensitizer (presently estimated in the range of 500-850 nm). Light in this spectral range and used intensities is not hazardous to the fetus or patient. The estimated light intensity to be delivered is in the range of 50-500 mW/1-5 min exposures, depending on the flexible light guide 62, the mode of illumination used, the nature of the target tissue and the objective of the treatment.

In one embodiment, the flexible light guide 62 of the therapeutic light system 60 is equipped with front-end optics. The flexible light guide 62 may be inserted to the target tissue or to its close proximity, according to the treated target and the objective of the treatment, and it may be inserted simultaneously with or following the insertion of the flexible drug delivery catheter 51 (see FIG. 1C).

Any suitable operating switch 64 may be used according to the invention such as foot-operated, hand-operated, motion-operated or computer-operated switches. In a preferred embodiment of the invention, the operating switch 64 for the therapeutic light system 60 is a foot-operated switch, most preferably a pedal, as shown in FIG. 1D. Such foot-operated switch enables a person who uses the endoscopic imaging photodynamic system of the invention, to operate the therapeutic light system 60 without the need to use his hands, which may be occupied by other components of the system.

FIG. 1E illustrates schematically one embodiment of the flexible service catheter 70 of the invention, adapted to contain the flexible drug delivery catheter 51 of the drug delivery module 50 and the flexible light guide 62 of the therapeutic light system 60. The latter are inserted into the flexible service catheter 70 to present the needle and tip of the flexible light guide, respectively, at the outlet orifice of the operative channel represented by 43 (see FIG. 1B).

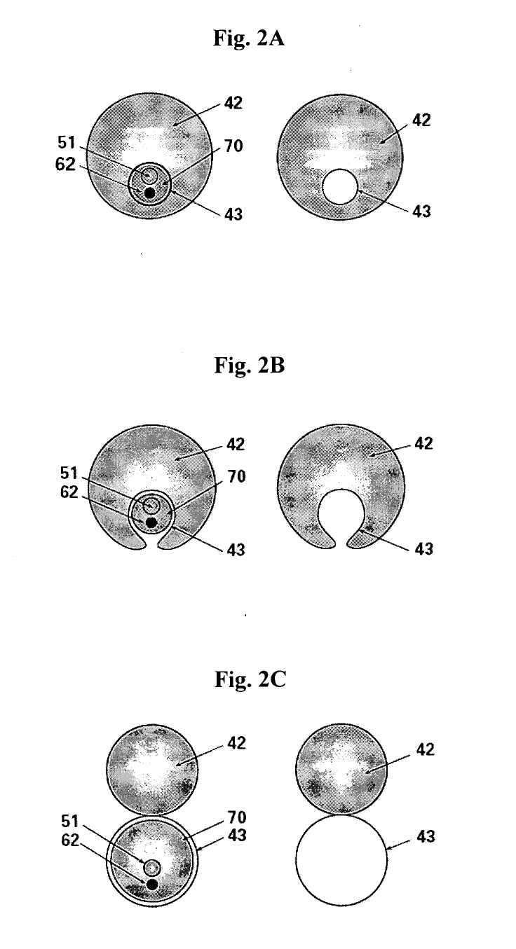

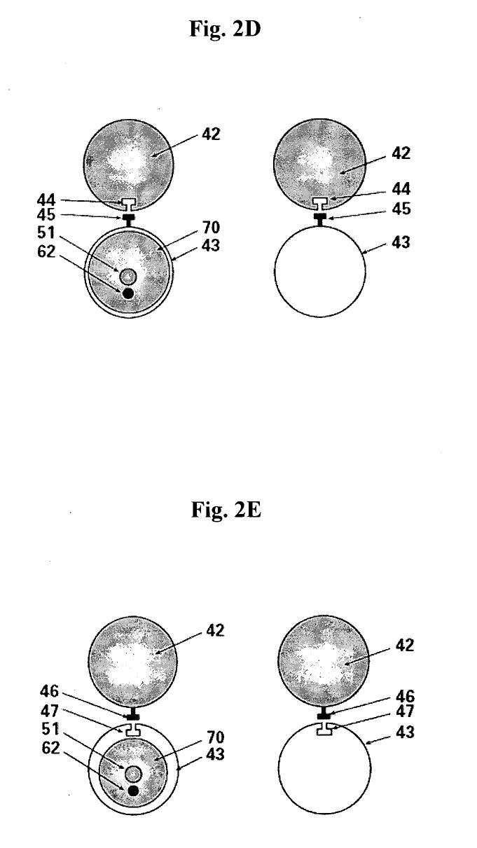

According to the invention, the real-time imaging component 40 comprises a flexible transducer 42 with an operative channel 43 through which the flexible service catheter 70 is inserted. Several possibilities are envisaged by the invention for positioning of the flexible transducer 42 and the operative channel 43 containing the inserted flexible service catheter 70. In principle, the operative channel 43 may be internal, namely, positioned within the transducer 42, or external, positioned in different positions, as depicted in FIGS. 2A-2E.

In FIG. 2A the operative channel 43 is positioned inside the flexible transducer 42. FIG. 2B illustrates the possibility wherein the operative channel 43 is positioned as a groove in the flexible transducer 42. In FIG. 2C the operative channel 43 is attached to the flexible transducer 42 along its full length. FIG. 2D illustrates the possibility wherein the operative channel 43 has connecting means 45 for attachment to a groove 44 of the flexible transducer 42. In FIG. 2E the flexible transducer 42 has connecting means 46 for attachment to a groove 47 of the operative channel 43.

In FIGS. 2A-2E, the left side presents the flexible drug delivery catheter 51 of the drug delivery module 50 and the flexible light guide 62 of the therapeutic light system 60 inserted into the flexible service catheter 70 contained within the operative channel 43.

When the operative channel 43 is positioned inside the flexible transducer 42, the size of both the operation channel 43 and of the flexible service catheter 70 is inherently predetermined by the size of the flexible transducer 42. When the operative channel 43 is external, as depicted in FIGS. 2C-2E, the diameter of the flexible service catheter 70 may be larger than the diameter of the flexible transducer 42.

The endoscopic imaging photodynamic therapy system (EIPS) of the invention is designed for optimal delivery of minimally invasive internal treatments by photodynamic means, by local or systemic photosensitizer administration, under controlled real-time imaging. The EIPS of the invention can be used in various medical applications which require tissue ablation and where PDT may be applied such as, but not limited to, for treatment of reproductive tract diseases, disorders or lesions; gastrointestinal tract lesions; cardiological diseases, e.g., atrial arrhythmia, atrial fibrillation and ventricular tachycardia; respiratory system diseases; urinary tract diseases; musculo-skeletal diseases; central nervous system (CNS) diseases; pre-malignant and malignant lesions; benign tumors such as benign prostatic hyperplasia (BPH) and angiomas; and malignant tumors and neoplasms in all organs, including the brain.

It is to be understood that all operations of the EIPS of the invention can also be software-controlled and operated, for example, by a personal computer or a dedicated device and these embodiments are encompassed by the present invention.

The EIPS illustrated in FIGS. 1A-1E and, in particular, the endoscopic assembly of FIG. 1A is an illustrative example for focused tissue ablation for a procedure in the female reproductive tract such as extrauterine pregnancy. However, the invention is not limited to this configuration, but it encompasses any modification thereof, particularly in the application adaptor 30, that can be modified and made appropriate for use in any other tissue.

In one preferred embodiment, the EIPS of the invention is for use in the treatment of diseases, disorders or lesions of the reproductive tract, particularly in the treatment of gynaecological diseases, disorders and abnormalities such as, but not limited to, extrauterine pregnancy (EUP), ovarian pathologies, uterine fibroids (intramural, submucose or subserous) and other uterine lesions and tumors, pelvic endometriosis, and cervical, vaginal or vulvar lesions.

In a more preferred embodiment, the EIPS of the invention is for use in the treatment of extrauterine pregnancy (EUP).

The description below illustrates the use of the EIPS of FIGS. 1A-1E for treatment of EUP by transvaginal focused tissue ablation by illumination of a photosensitizer drug in a target tissue, wherein the target tissue is the feto-placental unit in an ectopic location. Most ectopic pregnancies occur in the Fallopian tube and are known in the art as “tubal pregnancies”; however, implantation can also occur in the cervix, ovaries and abdomen.

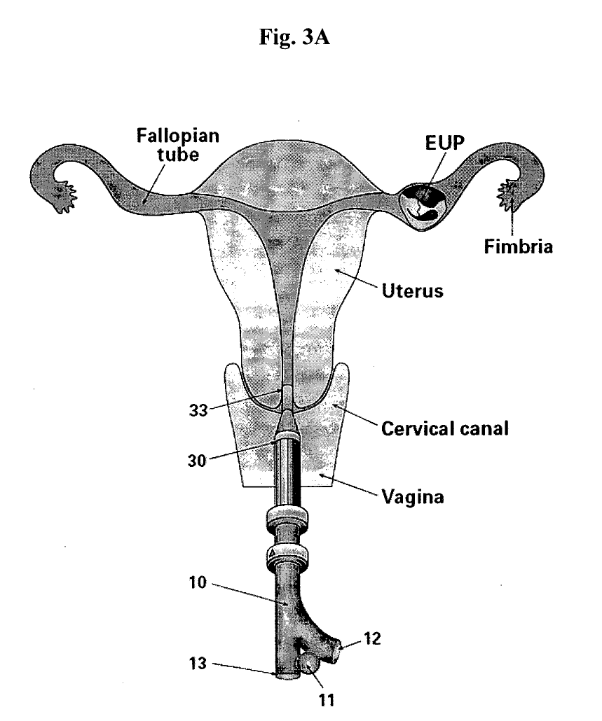

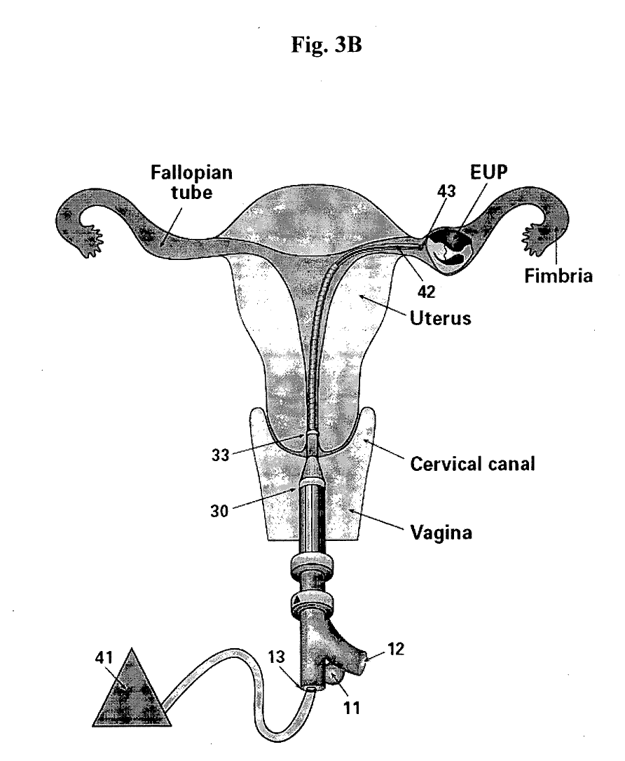

FIGS. 3A-3B show a schematic illustration of focused tissue ablation of a feto-placental unit in an ectopic location, using the endoscopic imaging photodynamic therapy system described in FIG. 1.

FIG. 3A depicts the transcervical controlled insertion of the flexible guide 33 of the application adaptor 30 of the EIPS. Fallopian tubes are shown and, on the right, an extrauterine pregnancy (EUP).

FIG. 3B depicts the tubal catheterization of the EIPS, namely, the vaginal insertion of the flexible transducer 42 containing the inserted operative channel 43 towards the EUP target. The imaging system is represented by 41 (see FIG. 1B). For treatment of EUP, the intervention is performed while the candidate patient is in a lithotomy position. In a preferred embodiment, the flexible transducer 42 of the imaging component 40 is inserted into one of the service openings 12/13 of the control handle 10 of the endoscopic assembly 100, which is transvaginally inserted into the cervical canal all the way to the target under real-time imaging guidance, e.g., ultrasound. The target EUP is examined by ultrasonography and US Doppler as needed or, as mentioned above, alternative imaging techniques may be used instead of ultrasonography.

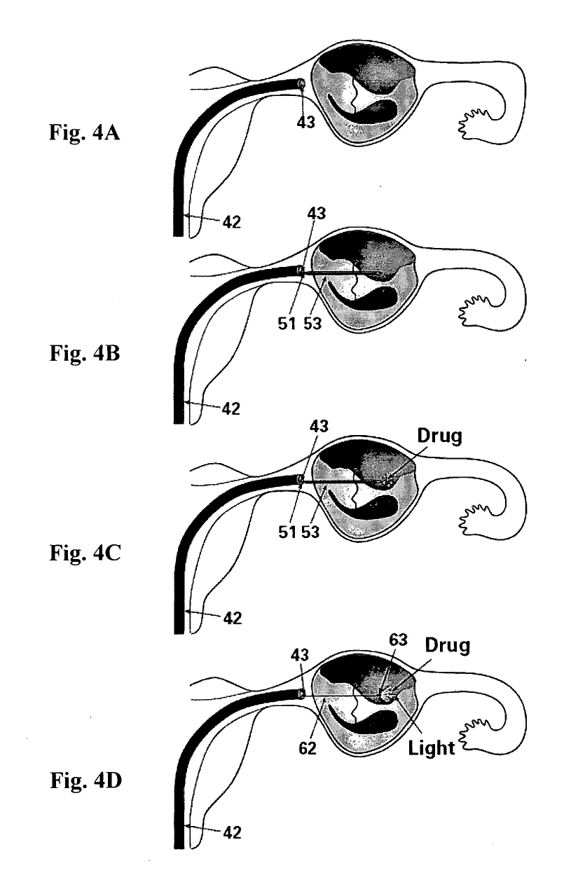

FIG. 4A-4D are illustrative of the next steps of the EUP intervention. The flexible service catheter 70 is next inserted into the operative channel 43 of the flexible transducer 42 of the imaging component 40 and further pushed towards the EUP target (FIG. 4A). The flexible service catheter 70 (not shown because it is inside 43) is positioned and aligned with the navigator dial 21 of the endoscopic assembly 100 under ultrasound guidance to the appropriate injection site. A catheter may also be inserted via the other one of the service openings 12/13 of the endoscopic assembly 100 and used for washing and aspiration of reproductive tract secretions and blood, as needed.

The flexible drug delivery catheter 51 of the drug delivery module 50 is inserted through the flexible service catheter 70 and guided to the target EUP, thus bringing the needle 53 to the target EUP to permit the photosensitizer drug (in an injectable form) injection by the delivery means 52 (FIG. 4B). When appropriate, the syringe needle 53 is inserted into the EUP (FIG. 4B). The therapeutic photosensitizer drug is then injected into the feto-placental unit (FIG. 4C). The drug delivery syringe 52 is attached to the external side of the flexible drug delivery catheter 51.

When appropriate, the flexible drug delivery catheter 51 of the drug delivery module is retracted and the flexible light guide 62 of the therapeutic light system 60 with an optic lens/diffuser 63 are inserted through the flexible service catheter 70 and placed in position (FIG. 4D). In one embodiment, the flexible drug delivery catheter 51 is not retracted and the flexible light guide 62 is inserted to the target tissue or to its close proximity simultaneously with the flexible drug delivery catheter 51.

FIG. 4D depicts therapeutic illumination with the flexible light guide 62 of the light source at the injection site of the photosensitizer drug at the EUP target. Following the appropriate time interval, the light is switched “on” with the foot-operated switch 64 (see FIG. 1D) for the planned amount of time required for inducing photodamage and tissue ablation in the target tissue. Upon completion of the illumination step, the EIPS is retracted from the patient body.

The device and technology described in FIGS. 3 and 4 refer to an intervention according to the invention in human females in which the real-time imaging component, using Doppler ultrasound technology, provides the means for real-time guidance and monitoring of the transvaginal intervention (including spatial orientation and blood flow). The vaginally-inserted flexible transducer and the flexible service catheter are appropriately presented for optimal relay of the streaming image to the external monitor of the real-time imaging component. The real-time imaging component continuously reports on the insertion process of the device, the location and identification of the feto-placental unit and its position within the Fallopian tube or other abnormally located position (e.g., uterine cornus or cervix), and is instrumental in the diagnosis of the patient and in the planning/execution of the treatment. In addition, the real-time imaging component continuously reports on the treatment progress and the treatment endpoints using present or future technologies that are adapted to the specific treatment.

The present invention further provides endoscopic methods for focused tissue ablation of a target tissue using an endoscopic imaging photodynamic therapy system according to the invention. In these methods, the photosensitizer drug may be injected either by local injection using a drug delivery module as described herein, or systemically, e.g., by intravenous infusion. The photosensitizer drug can be injected either before the EIPS is inserted or after the device is in place and ready for operation/illumination. In a preferred embodiment, the photosensitizer drug is administered by continuous infusion throughout the illumination step.

In one embodiment, the present invention provides an endoscopic method for focused tissue ablation of a target tissue of an individual using an endoscopic imaging photodynamic therapy system according to the invention, said method comprising:

(i) inserting the endoscopic assembly (a) into a cavity of the individual's body that leads to the target tissue;

(ii) inserting the flexible transducer of the real-time imaging component (b) through a service opening of the endoscopic assembly;

(iii) guiding the flexible transducer to the target tissue and locating the target tissue with the aid of said real-time imaging component;

(iv) inserting the flexible drug delivery catheter of the drug delivery module (d) through the operative channel of the flexible transducer, pushing towards the target tissue, and positioning and aligning the flexible drug delivery catheter with a navigator dial or computer-operated means under real-time imaging guidance to the appropriate injection site at or close to the target tissue;

(v) injecting a photosensitizer drug directly into or close to the target tissue with the drug delivery means;

(vi) retracting the flexible drug delivery catheter;

(vii) inserting the flexible light guide of the therapeutic light system (c) through the operative channel of the flexible transducer and positioning the flexible light guide adjacent to the target tissue;

(viii) delivering a therapeutic light dose to the target tissue, thus inducing photodamage and tissue ablation in the target tissue; and

(ix) retracting the endoscopic imaging photodynamic therapy system.

In another embodiment, the invention provides an endoscopic method for focused tissue ablation of a target tissue of an individual using an endoscopic imaging photodynamic therapy system according to the invention, said method comprising:

(i) inserting the endoscopic assembly (a) into a cavity of the individual's body that leads to the target tissue;

(ii) inserting the flexible transducer of the real-time imaging component (b) through a service opening of the endoscopic assembly;

(iii) guiding the flexible transducer to the target tissue and locating the target tissue with the aid of said real-time imaging component;

(iv) inserting the flexible drug delivery catheter of the drug delivery module (d) and the flexible light guide of the therapeutic light system (c) concomitantly through the operative channel of the flexible transducer, pushing towards the target tissue, and positioning and aligning the flexible drug delivery catheter with a navigator dial or computer-operated means under real-time imaging guidance to the appropriate injection site at or close to the target tissue;

(v) injecting a photosensitizer drug directly into or close to the target tissue with the drug delivery means;

(vi) positioning the flexible light guide adjacent to the target tissue;

(vii) delivering a therapeutic light dose to the target tissue, thus inducing photodamage and tissue ablation in the target tissue; and

(viii) retracting the endoscopic imaging photodynamic therapy system.

In one embodiment, the flexible drug delivery catheter of the drug delivery module and the flexible light guide of the therapeutic light system are inserted through the flexible service catheter inserted into the operative channel of the flexible transducer.

As used herein, the term “cavity of an individual's body” refers to any cavity, lumen or other inner space of an organ or tissue into which an endoscopic system of the invention can be inserted in order to effect the ablation treatment in the target tissue.

The invention further provides an endoscopic method for focused tissue ablation of a target tissue of an individual using an endoscopic imaging photodynamic therapy system according to the invention comprising an endoscopic assembly (a), a real-time imaging component (b), and a therapeutic light system (c), said method comprising:

(i) injecting/infusing a photosensitizer drug systemically to the individual;

(ii) inserting the endoscopic assembly (a) into a cavity of the individual's body that leads to the target tissue;

(iii) inserting the flexible transducer of the real-time imaging component (b) through a service opening of the endoscopic assembly;

(iv) guiding the flexible transducer to the target tissue and locating the target tissue with the aid of said real-time imaging component;

(v) inserting the flexible light guide of the therapeutic light system (c) through the operative channel of the flexible transducer, pushing towards the target tissue, and positioning the flexible light guide adjacent to the target tissue;

(vi) delivering a therapeutic light dose to said target tissue, thus inducing photodamage and tissue ablation in the target tissue; and

(vii) retracting the endoscopic imaging photodynamic therapy system.

In a preferred embodiment, the invention provides a method for focused tissue ablation of a feto-placental unit in an extrauterine pregnancy (EUP) in a female patient using an endoscopic imaging photodynamic therapy system comprising an endoscopic assembly, a real-time imaging component, a drug delivery module, and a therapeutic light system, said method comprising:

(i) inserting the endoscopic assembly into the vaginal tract of the female patient with EUP;

(ii) inserting a flexible transducer with an operative channel of the real-time imaging component through a service opening of the endoscopic assembly;

(iii) guiding the flexible transducer to the target EUP through the vaginal tract and locating the target EUP with the aid of said real-time imaging component;

(iv) inserting a flexible drug delivery catheter of the drug delivery module through the operative channel of the flexible transducer, pushing towards the target EUP, and positioning and aligning the flexible drug delivery catheter with a navigator dial or computer-operated means under real-time imaging guidance to the appropriate injection site at or close to the target EUP;

(v) injecting a photosensitizer drug directly into the target EUP with a drug delivery means of the drug delivery module;

(vi) retracting the flexible drug delivery catheter;

(vii) inserting a flexible light guide of the therapeutic light system through the operative channel of the flexible transducer, pushing towards the target EUP, and positioning the flexible light guide adjacent to the target EUP;

(viii) delivering a therapeutic light dose to said target EUP, thus inducing photodamage and tissue ablation of the feto-placental unit in the target EUP; and

(ix) retracting the endoscopic imaging photodynamic therapy system.

In another preferred embodiment, the invention provides a method for focused tissue ablation of a feto-placental unit in an extrauterine pregnancy (EUP) in a female patient using an endoscopic imaging photodynamic therapy system comprising an endoscopic assembly, a real-time imaging component, a drug delivery module, and a therapeutic light system, said method comprising:

(i) inserting the endoscopic assembly into the vaginal tract of the female patient with EUP;

(ii) inserting a flexible transducer with an operative channel of the real-time imaging component through a service opening of the endoscopic assembly;

(iii) guiding the flexible transducer to the target EUP through the vaginal tract and locating the target EUP with the aid of said real-time imaging component;

(iv) inserting a flexible drug delivery catheter of the drug delivery module and a flexible light guide of the therapeutic light system concomitantly through the operative channel of the flexible transducer, pushing towards the target EUP, and positioning and aligning the flexible drug delivery catheter with a navigator dial or computer-operated means under real-time imaging guidance to the appropriate injection site at or close to the target EUP;

(v) injecting a photosensitizer drug directly at the target EUP with a drug delivery means of the drug delivery module;

(vi) positioning the flexible light guide adjacent to the target EUP;

(vii) delivering a therapeutic light dose to said target EUP, thus inducing photodamage and tissue ablation of the feto-placental unit in the target EUP; and

(viii) retracting the endoscopic imaging photodynamic therapy system.

The Examples hereinbelow were carried out in a rat model of EUP. Since EUP is not a natural or inducible condition in laboratory animals, it was necessary to find a suitable model for this study. The rat model selected in this study for in utero photodynamic feto-placental ablation yielded encouraging results, showing promise for clinical EUP treatment, in spite of the fact that the rat was exposed to far greater risks than foreseen in pregnant women: (i) the rat underwent abdominal surgery with trans-uterine PDT, whereas in pregnant women a minimally invasive trans-vaginal approach is suggested; (ii) a single, selected feto-placental unit (in a litter of 7-12) has been safely ablated without detectable lateral or collateral damage, while the ectopic pregnancy in women will be the sole target, without need to spare any neighboring embryos; (iii) there was no alternative to trans-uterine administration of PDT in the rat while the minimally invasive trans-vaginal approach proposed for EUP-PDT in women will allow direct delivery of the treatment into the placenta (by endoscopic means); and (iv) using the rat uterus as model for the Fallopian tube (that is not homologous), while reasonable, entails higher risks when preservation of functional integrity following PDT is obligatory, especially during muscle stress expected during parturition.

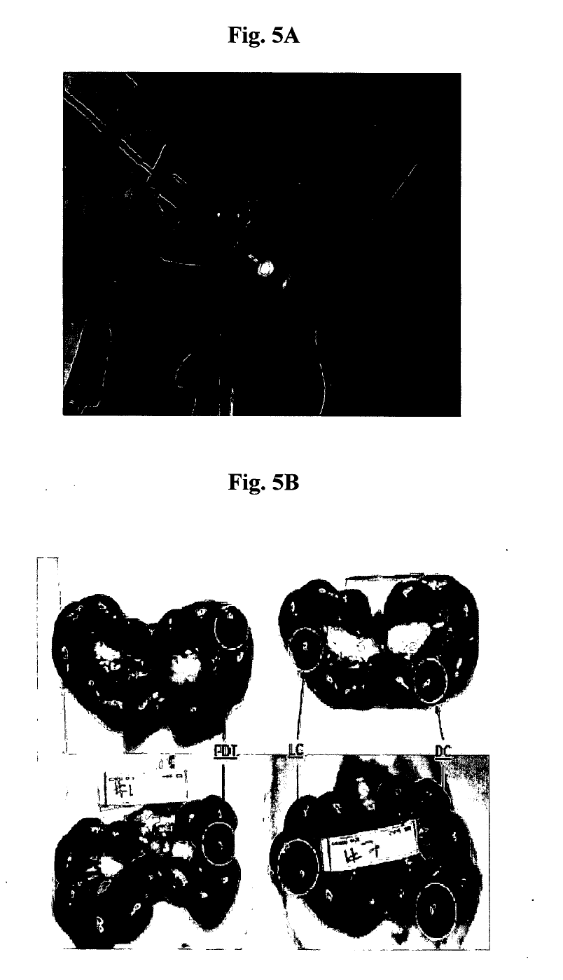

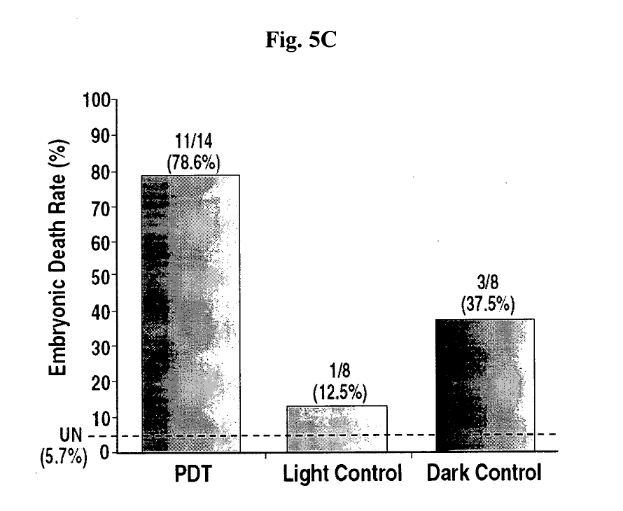

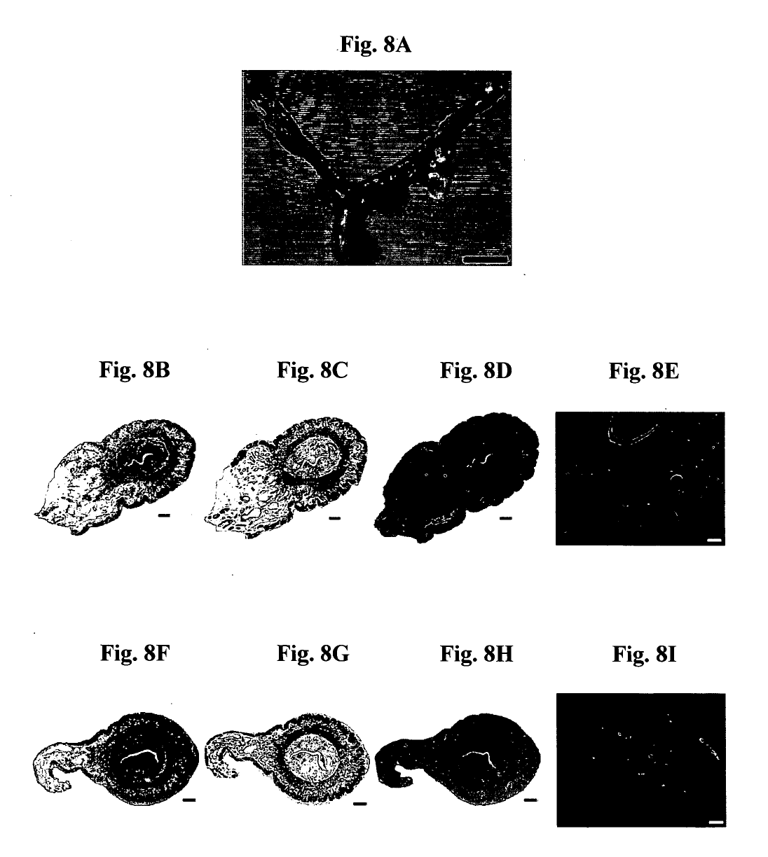

In summary, the intervention in the rat is significantly more severe, morbid and potentially damaging to the uterus than the proposed trans-vaginal approach proposed according to the invention for female patients. It is important to note the normal completion of gestation (evidence for uterine integrity as well as normal smooth muscle strength) with conservation of fertility potential, in all the tested rats (FIGS. 5B, 6-8). Histological evidence of the post-PDT rat uterus points to: (i) the absence of adhesions or scarring, suggesting a reduced probability for an additional EUP (in humans) as revealed by pan-cytokeratin staining; (ii) structural integrity of the uterine wall with no rupture following treatment and consequent parturition (as shown by SMA staining); and (iii) absence of vascular malformations, thrombosis or other long term vascular effects (vWF staining) (FIG. 8). The above results indicate that placental PDT will not have a severely deleterious effect on the Fallopian tube in EUP patients.

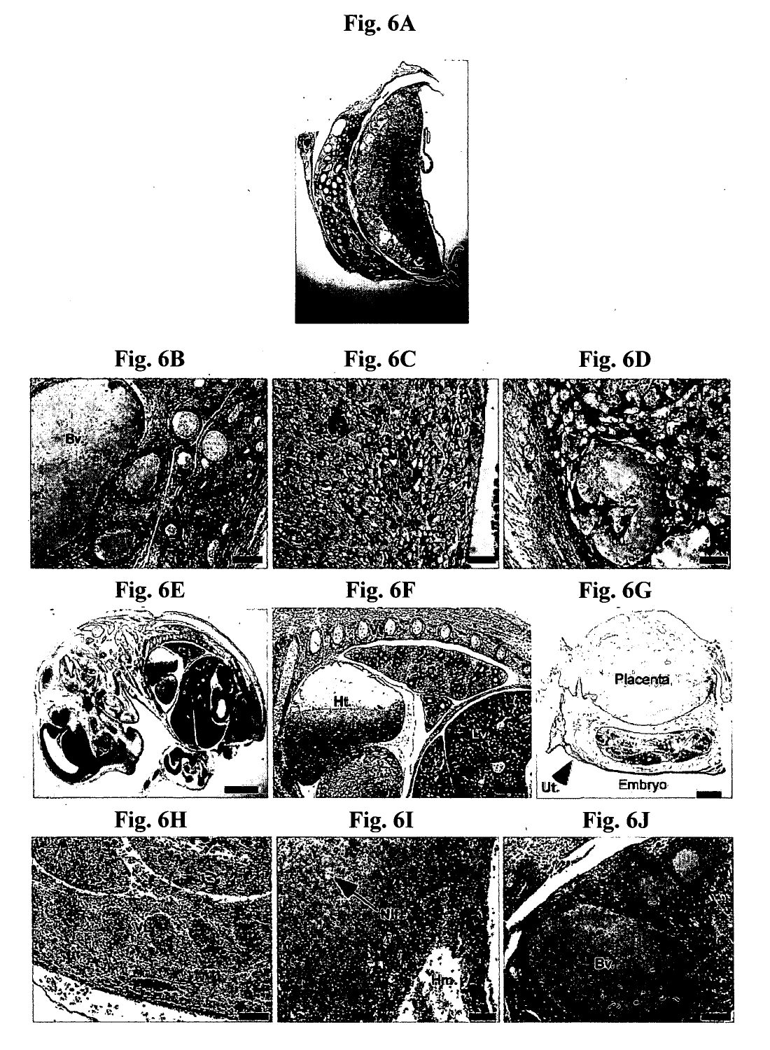

As for the photodynamic damage inflicted upon feto-placental units, histological analysis of feto-placental units subjected to PDT demonstrated dramatic effects in the severely damaged targeted feto-placental units, while the uterine tissues remained intact (FIG. 6). These observations are indicative of the localized nature of this treatment protocol as well as of the absence of photosensitizer in maternal tissues. Interestingly, there were cases where feto-placental units treated by PDT showed only partial placental damage while pregnancy was terminated (data not shown). This suggests that partial damage of the placenta may be sufficient but the extent of damage required for a successful outcome may vary. While in PDT of solid tumors the drug is systemically administered, local interstitial drug administration into the placenta is advised here. The damage inflicted to the placenta could, therefore, be a mixture of simple injury, direct cellular phototoxicity as well as photodamage to vascular components following dissemination of drug into the feto-placental circulation. The pathological analysis reveals evidence for both cellular and vascular effects (FIGS. 6H-J). Pathological analysis of feto-placental controls (UN, LC and DC) in which pregnancy was terminated demonstrates varying degrees of necrosis (10-50%), suggesting damage originating from causes other than the photodamage induced by PDT, such as mechanical damage and/or dark toxicity of the drug to the feto-placental unit.

The size of the rat embryonic sac at the time of the photodynamic intervention (approximately 2 cm in diameter at E14) is similar to the size of human extrauterine pregnancies eligible for conservative treatment (<4 cm in diameter). In contrast to humans where pregnancy termination results in abortion, in rodents mortality of embryos culminates with gradual absorption in utero, permitting their post treatment examination.

As stated above, the only previously reported attempt to address the problem of EUP by means of PDT (Yang et al., 1993, 1994) was in the early 90's employing a very different approach. The photosensitizer 5-ALA, which suffers from both a slow clearance rate as well as inferior photochemical properties, was systemically administered to the pregnant rat on E10 and the entire uterine horn was illuminated up to 30 min later, resulting in massive endometrial ablation and loss of all embryos in the treated uterine horn when examined on E17. Furthermore, one third of the treated animals did not conceive following PDT and the unlikely re-conception in the treated uterine horns was not reported. Pregnancy termination in this case was probably the result of an anti-endometrial effect and not due to direct damage to any single specific embryo. No subsequent follow up of these studies was reported by these or other investigators. Subsequent reports of this group focused on endometrial ablation by 5-ALA-PDT as a potential treatment for endometriosis (Krzemien et al., 2002).

The procedure disclosed in the Examples hereinafter overcame the adverse effects (endometrial damage, infertility, systemic photosensitivity) caused by the above attempts of pregnancy termination by PDT with 5-ALA. The novel photosensitizer used in the examples herein (PMRDA), like other bacteriochlorophyll-derived PDT drugs, has several important advantages over the previously used 5-ALA: (i) spectral absorption in the NIR, allowing deeper tissue penetration of light with larger treatment volume; (ii) PMRDA can be photosensitized immediately upon administration, not requiring prior metabolic conversion into a photosensitive form like 5-ALA, allowing intra-operative application and shortening the procedure; (iii) rapid clearance results in little to no skin phototoxicity (not relevant to this procedure because of local administration of the drug); and (iv) local administration of low doses as performed in this study reduces eventual photodamage to neighboring tissues and organs by restricting damage solely to the photosensitizer-containing tissue and sparing damage from the trans-illuminated uterine wall.

The nearly 80% success rate of pregnancy termination with PDT disclosed in the Examples herein is consistent with the range of success rates obtained with PDT in several tumor models in mice and rats (69-92% tumor free following a single treatment session) using other bacteriochlorophyll derived photosensitizers including WST09 and WST11 (Zilberstein et al., 2001; Koudinova et al., 2003; Preise et al., 2003; Mazor et al., 2004; Plaks et al., 2004; Kelleher et al., 2003; Kelleher et al., 2004).

Potentially, the gynaecological applications of PDT with bacteriochlorophyll derived photosensitizers like PMRDA and others may extend beyond EUP treatment, e.g., pelvic endometriosis, uterine fibroid tumors, ovarian, vulvar, vaginal and cervical lesions, pre-cancerous, and malignant tumors of the reproductive system. As recently reviewed by Allison et al. (2005), the major hurdles in PDT applications in gynecology with 5-ALA, Foscan® or Photofrin® as photosensitizers are insufficient depth of treatment and skin phototoxicity. Applying Pd-bacteriochlorophyll based photosensitizers holds great promise of circumventing these shortcomings due to their superior properties, as demonstrated in cancer therapy (Koudinova et al., 2003; Weersink et al., 2005).

In summary, this study demonstrates for the first time that placental-PDT is an applicable, highly efficient, fertility-preserving intervention, able of selective ablation of a specific rat feto-placental unit, without significant local or systemic adverse effects, and indicates its possible clinical translation for treatment of women suffering from extrauterine pregnancy, offering a highly controlled, local, short and cost effective minimally-invasive modality.

The proposed intervention procedure according to the invention in the rat model of extrauterine pregnancy is local, highly accurate allowing for specific ablation of a selected embryo in the litter, and is short (a few minutes). In addition, the provided method has no deleterious effects on the treated dam and neighboring embryos with complete preservation of fertility.

In one embodiment of the present invention, the photosensitizer drug and the therapeutic light are delivered locally into the feto-placental unit by the endoscopic imaging photodynamic therapy system of the invention, thereby enabling a focused ablation of the abnormal pregnancy, without causing mechanical damage to the Fallopian tube. Thus, the method of the present invention offers higher success rates and maximal fertility preservation. In addition, the separation of fetal from systemic maternal blood systems offers a potential confinement of the PDT agent to the target (placenta), thus preventing maternal exposure and adverse effects.

Any photosensitizer suitable for PDT can be used in the present invention such as the approved photosensitizers Photofrin and 5-aminolevulinic acid (5-ALA), the second generation photosensitizers in the investigational stage and, particularly, the chlorophyll and bacteriochlorophyll derivatives disclosed in the following patents and patent applications of the applicant: EP 0584552, WO 97/19081, WO 00/33833, WO 01/40232, WO 2004/045492 and WO 2005/120573, all incorporated herewith by reference as if fully disclosed herein. The invention will now be illustrated by the following non-limiting examples.

### EXAMPLES

Materials and Methods (i) Animals. Pregnant 10-week old female Wistar rats (litter sizes of 10±2) (Harlan Laboratories, Rehovot, Israel) were delivered to the lab on embryonic day 7-12 (E7-E12) and allowed acclimatization of at least 48 h. All animal protocols were approved by the Weizmann Institutional Animal Care and Use Committee (IACUC).

(ii) Photosensitizer. Palladium 3¹-oxo-15 -methoxycarbonylmethylrhodobacterio-chlorin-13¹,17³-di(2-N2-dimethylaminoethyl) amide (PMRDA), a water-soluble positively charged Pd-bacteriochlorophyll derivative disclosed in WO 2005/120573, was either synthesized in the inventor's laboratory by known methods or kindly provided by Steba Laboratories Ltd. (Israel), and used as a photosensitizer. The PMRDA was obtained as powder and kept under Argon (Ar) at −20° C. in the dark. Thereafter it was dissolved in methanol (MeOH) and, following spectrophotometric determination of concentration, was divided into aliquots, dried under Ar and kept at −20° C. in the dark until use.

(iii) Light source. A 1W 755 nm laser (CeramOptec, Bonn, Germany) was used. Light was delivered via a diffuser tipped optic fiber (Medlight S.A., Ecublens, Switzerland).

(iv) In vivo PDT protocol. A pregnant rat (E14) was anesthetized by isoflurane inhalation using the IMPAC6 system (VetEquip Inc., Pleasanton, Calif., USA) and placed on its back; the lower abdomen was shaved, washed with 70% ethanol and draped with sterile gauze. A midline incision (approximately 2 cm in length) in the lower abdomen was performed by cutting the skin and abdominal-muscle layers separately. Using round forceps, the uterus was gently drawn out from the abdominal cavity and placed on the gauze. The exposed uterus was continuously irrigated with sterile 0.9% NaCl solution during the procedure (for layout, see FIGS. 5A, 5B). One embryo was randomly selected for treatment and positioned with the placenta facing up. The laser beam was then directed to the exposed placental pole and the beam positioned for subsequent therapeutic illumination (6 mm light spot diameter) using the built-in visible (630 nm, 3 mW) aiming beam. No shielding of neighboring embryos was required. The photosensitizer PMRDA (50 μg/30 μl sterile saline) was delivered by trans-uterine injection (30G, 0.3 ml, insulin syringe) into the placenta. Following a drug/light time interval (DLTI) of 120 sec, illumination (100 mW/cm²) was delivered (300 sec) to the placenta in a trans-uterine manner from above, perpendicular to the uterus (FIG. 5A). Upon completion of the PDT protocol, the uterus was eased back into the abdominal cavity. The abdominal facia was sutured using sterile 4-0 braided silk thread and the skin was clipped using 9 mm surgical clips. Anesthesia was discontinued and the rat was placed in a cage and closely monitored for at least 48 h. Animals received analgesia (Oxycod®, 6 ml/l) in their drinking water for at least 48 h. Controls: Dark control (DC): placental administration of photosensitizer without illumination. Light control (LC): placental sham injection of sterile vehicle (saline), followed by illumination with the respective light dose. Untreated control (UN): all untreated fetoplacental units in a treated rat.

(v) Euthanasia. Animals were sacrificed by CO₂ inhalation, in accordance with IACUC guidelines.

(vi) Post PDT fertility assessment. Treated rats were allowed to reach parturition and pup weaning (P21). Fertile males were then introduced into the cages and rats were allowed to mate until pregnancy could be verified by daily vaginal smears and palpation. Pregnancy in both uterine horns was verified by magnetic resonance imaging (MRI) and/or by animal sacrifice and macroscopic examination. To fully prove fertility of rats post PDT, the MRI examined rats were allowed to bear the second litter to parturition and pup-weaning.

(vii) In vivo MRI of pregnant rats. The pregnant female rats (E12-E16) were anesthetized by intra-peritoneal injection of ketamine/diazepam (1:1 v/v). MR images were acquired on a horizontal 4.7 T Bruker-Biospec spectrometer using a volume coil. Gradient echo 3D T₂ weighted sequence was acquired with 10 ms repetition time, 3.5 ms echo time, 5° flip angle, 2 scans, 128×128×128 matrix size, 8×4×4 cm³ field of view.

(viii) Histology. Following animal sacrifice, the abdomen was surgically opened, the uterus was tied-off at the cervix, removed and placed in Carnoy's fixative (6:3:1 ethanol, chloroform and acetic acid, by vol.), or Tris-buffered Zn fixative (2.8 mM calcium acetate, 22.8 mM zinc acetate, and 36.7 mM zinc chloride in 0.1M Tris buffer, pH 7.4) for 48-72 h. Fixed tissue samples were placed in 70% ethanol, paraffin embedded and slides were prepared. Slides were stained with hematoxylin and eosin (H&E) Immunohistochemical staining of the specific cell markers was done using the following antibodies: sheep anti-von Willebrand Factor (vWF)] (Serotec, Oxford, UK), mouse anti-pancytokeratin (clone AE1/AE3, Zymed, San-Francisco, USA) and mouse anti-human smooth muscle actin (SMA) (a SMC marker) (clone 1A4, Serotec, Oxford, UK).

(ix) Light microscopy. Microscopic images were taken using a Nikon ECLIPSE E600 microscope equipped with a Nikon DXM1200F digital camera (Nikon Instech Co., Kanagawa, Japan).

(x) Photography. Macroscopic images were taken using a Fujifilm MX-2900 ZOOM digital camera (Fuji Photo Film Co., Tokyo, Japan).

### Example 1

### The Response of a Selected Fetoplacental Unit in the Pregnant Rat to Local PMRDA-Based PDT

In order to assess the feasibility of employing PDT for the ablation of a single embryo in a litter, a novel procedure of placental-PDT with PMRDA as photosensitizer was developed and tested.

The photodynamic treatment protocol was carried out in the pregnant rat on E14 (see Materials and Methods and layout, FIG. 5A). Upon surgical access, feto-placental units were subjected to PDT by local intra-placental injection of PMRDA followed by illumination. Macroscopic analysis and tissue sampling for histology were performed on E16. The PDT-ablated feto-placental unit appeared discolored and shrunken, undergoing uterine absorption whereas control embryos appeared normal and unaffected in most cases (FIG. 5B). In some cases where death was observed in control groups (untreated (UN), light control (LC) and dark control (DC)) the dead feto-placental units were partially or completely shrunken and disintegrated. In inconclusive cases, the surgical exposure of the embryonic sac after fixation allowed for a more detailed examination of the state of the feto-placental unit. The entire uteri were then excised, fixed and samples taken for histological examination as detailed below (FIG. 6).

Photodynamic treatment of a single embryo in the pregnant rat with PMRDA resulted in pregnancy termination in 11/14 embryos (78.6%). In control rats (one LC embryo and one DC embryo per litter, one treated embryo per uterine horn), embryo mortality rates were 1/8 (12.5%) for LC and 3/8 (37.5%) for DC. In a total of 22 litters (14 PDT and 8 control rats) the death rate of UN embryos was 13/230 (5.7%) (FIG. 5C).

These results clearly demonstrate that the developed photodynamic feto-placental ablation procedure can induce loss of the targeted embryo (terminating the pregnancy) without significantly affecting other embryos in the litter. Furthermore, this protocol did not result in noticeable morbidity to the treated pregnant rat and appeared efficient and reproducible, justifying further examination.

### Example 2

### Histological Analysis of the Response of Rat Feto-Placental Units to Local in Utero PDT

The fixed feto-placental units with the surrounding uterine wall were next examined histologically. FIGS. 6A-6F depict the histology of a normal untreated (E16) placenta and fetus. The normal placenta appears intact, with the following discernable features: (i) a heavily vascularized pregnant uterine wall (FIG. 6B); (ii) labyrinth layer, where fetal blood vessels cross maternal blood pools and exchange takes place, with fetal nucleated red blood cells (rbc) marking the fetal vasculature (FIG. 6C); (iii) spongiotrophoblast layer, with its characteristic ‘hollow-cell’ appearance (FIG. 6D); and (iv) the fetus, with normal well defined anatomy (FIG. 6E), such as vertebrae, heart, lungs and liver (FIG. 6F).

In post-PMRDA-PDT placentas severe damage was observed. A fully necrotic embryo was observed, presenting ill-defined structures, in a state of disintegration and necrosis, undergoing absorption (FIGS. 6G-6H). The placenta featured edema, congestion and moderate but extensive necrosis, involving approximately 70% of its mass (FIGS. 6I-6J). The surviving feto-placental LC and DC controls presented normal histology featuring no pathological findings. In cases where feto-placental controls were fatally damaged, histological examination revealed 10-50% necrosis (data not shown).

Importantly, the uterus adjacent to the treated placenta appeared viable (FIG. 6G) with minimal to mild multi-focal neutrophylic infiltration (FIG. 6I).

In conclusion, placental PDT is shown to induce a high rate of severe and significant photodamage in placental and embryonic tissues, which includes various degrees of hemorrhage (vascular damage), inflammation (immune response) and necrosis (cellular damage) of embryo and placental components, while sparing the uterine tissues from discernable damage. The absence of any apparent injury or damage to the uterus lead us to further examine the functional integrity of the reproductive system following PDT.

### Example 3

### Successful in vivo Pregnancy Termination by Placental PMRDA-PDT has No Deleterious Effects on Pregnancy Outcome and Subsequent Fertility

In order to examine the possible functional effects of PMRDA-PDT on the fertility of treated pregnant rats, two parameters were examined: (i) the ability of treated rats to achieve successful parturition of the remaining pups of the litter (indicative of the functional integrity of the uterine wall) and (ii) the ability of the post-PDT rat to conceive with implantation in the photodynamically treated uterine horn (indicative of uterine patency). To this end, six pregnant rats were treated and allowed to achieve parturition: four received PDT and two the respective control manipulations (one DC and one LC embryo per rat, one treatment per uterine horn). All six rats achieved normal parturition, delivering 6 to 11 pups without post-parturition maternal or pup mortality. The newborn pups were followed till P21 (weaning) and found to develop normally. This result suggested that placental PDT of a selected single feto-placental unit has no adverse effect on the remaining litter and subsequent parturition.

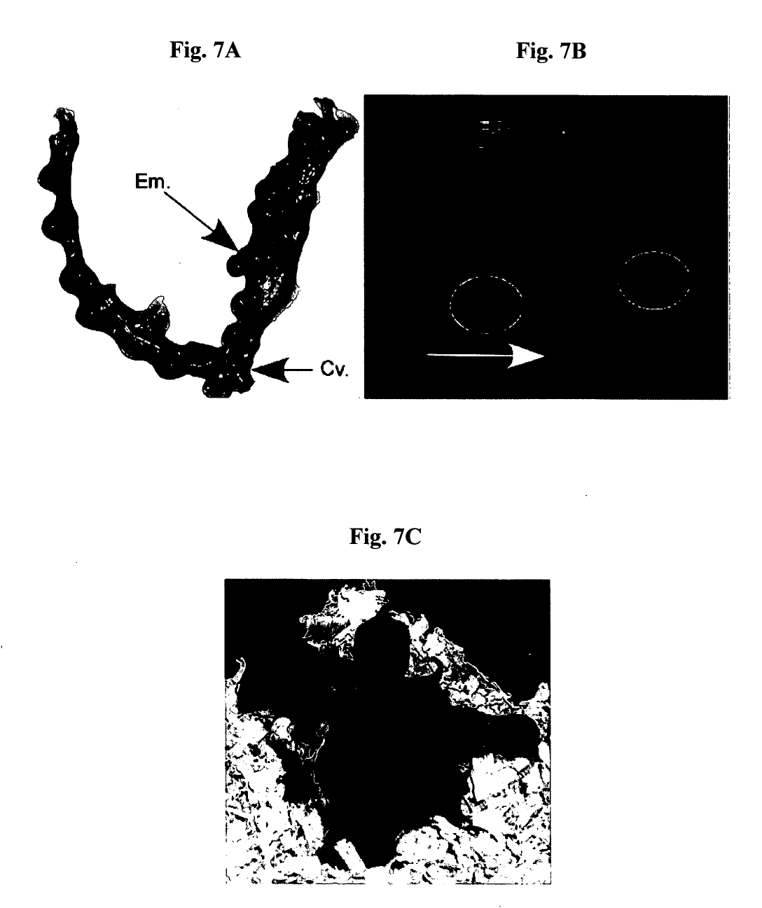

Following pup separation (P21), the resumption of normal estrus cycle in the rats was examined by sequential vaginal smearing. Once estrus cycle commenced, a fertile male was introduced into the cage and rats were allowed to mate, with the aim of examining these post-PDT rats for their ability to conceive and achieve parturition. Results were conclusive, with all six rats pregnant (litter sizes 6-11). Once pregnancy was confirmed, the rats were subjected to MRI examination in order to verify ongoing pregnancy in both treated and untreated uterine horns (5/6 rats were examined by MRI) (FIG. 7B). The MRI results were further verified by rat sacrifice and visual inspection of the uterus in three rats (two following placental PDT and one following control manipulations) (FIG. 7A). The remaining three rats were allowed to carry their pregnancy to term. Pups, dams and maternal care were followed as described above and found to be normal (FIG. 7C). These experiments demonstrated that following local in vivo placental PDT with PMRDA the fertility of the rats is preserved, specifically without compromising the ability of the PDT-treated uterine horn to re-implant.

### Example 4

### Following Treatment and a Parturition-Pregnancy-Parturition Cycle Uteri Retain Structural Integrity

Following verification of the functional integrity of post-PDT rat uteri, histological analysis was conducted in order to identify possible long-term damage resulting from the treatment. For this purpose, uteri in post-PDT and control manipulated rats were examined approximately 10 days after parturition (17-18 days after treatment). The respective uteri were stained by standard H&E (FIGS. 8B and 8F) as well as by anti-SMA antibody (a SMC marker, indicative of uterine wall integrity) (FIGS. 8C and 8G), anti-pan-cytokeratin antibody (an epithelial cell marker, indicative of endometrial integrity and absence of adhesions) (FIGS. 8D and 8H) and for vWF factor (an endothelial cell marker, illustrating blood vessel integrity) (FIGS. 8E and 8I). Uteri featured mild to moderate organizing hemorrhages in the endometrium and mesometrium, typical of post-gravid uteri (uteri after parturition). The only type of lesion found was the presence of a low number of hemosiderin-laden macrophages (siderophages) in the endometrium (where such a presence is normal) and in the mesometrium. This finding (siderophages) was highest in the DC uterus and milder in the post PDT uterus. No necrotic areas were found in any of the uteri inspected. The respective histological sections and various stains are shown in FIGS. 8A-8I.

### REFERENCES

Allison R., Cuenca R., Downie G., Randall M., Bagnato V., Sibata C., PD/PDT for gynecological diseases. A clinical review, Photodiagnosis and Photodynamic Therapy, 2005, 2, 51-63

Kelleher D. K., Thews O., Scherz A., Salomon Y., Vaupel P., Combined hyperthermia and chlorophyll-based photodynamic therapy: tumour growth and metabolic microenvironment, Br J Cancer, 2003, 89, 2333-2339

Kelleher D. K., Thews O., Scherz A., Salomon Y., Vaupel P., Perfusion, oxygenation status and growth of experimental tumors upon photodynamic therapy with Pd-bacteriopheophorbide, Int J Oncol, 2004, 24, 1505-1511

Koudinova N. V., Pinthus J. H., Brandis A., Brenner O., Bendel P., Ramon J., Eshhar Z., Scherz A., Salomon Y., Photodynamic therapy with Pd-Bacteriopheophorbide (TOOKAD): Successful in vivo treatment of human prostatic small cell carcinoma xenografts, Int J Cancer, 2003, 104, 782-789

Krzemien A. A., Van Vugt D. A., Fletcher W. A., Reid R. L., Effectiveness of photodynamic ablation for destruction of endometrial explants in a rat endometriosis model, Fertil Steril, 2002, 78, 169-175

Mazor O., Brandis A., Plaks V., Neumark E., Rosenbach-Belkin V., Salomon Y., Scherz A., WST11, A novel water-soluble bacteriochlorophyll derivative; cellular uptake, pharmacokinetics, biodistribution, and vascular targeted photodynamic activity using melanoma tumors as a model, Photochem Photobiol., 2005, 81, 342-351

Plaks V., Koudinova N., Nevo U., Pinthus J. H., Kanety H., Eshhar Z., Ramon J., Scherz A., Neeman M., Salomon Y., Photodynamic therapy of established prostatic adenocarcinoma with TOOKAD: a biphasic apparent diffusion coefficient change as potential early MRI response marker, Neoplasia, 2004, 6, 22

Preise D., Mazor O., Koudinova N., Liscovitch M., Scherz A., Salomon Y., Bypass of tumor drug resistance by antivascular therapy, Neoplasia, 2003, 5, 475-480

Reid R. L., Yang J. Z., Krzemien A., Melchior M. F., Van Dijk J. P., Hahn P. M., Van Vugt D. A., Greer P. A., Dickson E. F., Pottier R. H., Potential application of PDT for treatment of endometriosis and ectopic pregnancy: Animal models. In Photomedicine in Gynecology and Reproduction. Basel: Karger., 2000, 302-307

Weersink R. A., Forbes J., Bisland S., Trachtenberg J., Elhilali M., Brun P. H., Wilson B. C., Assessment of cutaneous photosensitivity of TOOKAD (WST09) in preclinical animal models and in patients, Photochem Photobiol, 2005, 81, 106-113

Yang J. Z., Van Vugt D. A., Kennedy J. C., Reid R. L., Evidence of lasting functional destruction of the rat endometrium after 5-aminolevulinic acid-induced photodynamic ablation: prevention of implantation, Am J Obstet Gynecol, 1993, 168, 995-1001

Yang J. Z., Van Vugt D. A., Melchior M. F., Hahn P. M., Reid R. L., Photodynamic ablation of early pregnancy in the rat with 5-aminolevulinic acid: a potential new therapy for tubal ectopic pregnancy in the human, Fertil Steril, 1994, 62, 1060-1065

Yang J. Z., Van Dijk-Smith J. P., Van Vugt D. A., Kennedy J. C., Reid R. L., Fluorescence and photosensitization of experimental endometriosis in the rat after systemic 5-aminolevulinic acid administration: a potential new approach to the diagnosis and treatment of endometriosis, Am J Obstet Gynecol, 1996, 174, 154-160

Zilberstein J., Schreiber S., Bloemers M. C., Bendel P., Neeman M., Schechtman E., Kohen F., Scherz A., Salomon Y., Antivascular treatment of solid melanoma tumors with bacteriochlorophyll-serine-based photodynamic therapy, Photochem Photobiol, 2001, 73, 257-266

1. An endoscopic imaging photodynamic therapy system for focused tissue ablation of a feto-placental unit in an extrauterine pregnancy (EUP) by illumination of a photosensitizer drug in said feto-placental unit, said system comprising an endoscopic assembly designed for entry into a vaginal tract of a female patient, a real-time imaging component for locating the feto-placental unit and monitoring the ablation intervention, a therapeutic light system and a drug delivery module, wherein said imaging component comprises a flexible transducer with an operative channel for insertion of a flexible light guide of the therapeutic light system and a flexible drug delivery catheter of the drug delivery module.

2. The endoscopic imaging photodynamic therapy system of claim 1, wherein:
(a) said endoscopic assembly comprises a control handle, an operation handle and an application adaptor;
(b) said real-time imaging component comprises means for guidance for location of said feto-placental unit and monitoring of the ablation intervention in said feto-placental unit, and a flexible transducer with an operative channel;
(c) said therapeutic light system consists of a light source, a flexible light guide and an operating switch for the light system; and
(d) said drug delivery module comprises a flexible drug delivery catheter adapted for injecting a photosensitizer drug to the feto-placental unit, a drug delivery means and a photosensitizer drug in an injectible form.

3. The endoscopic imaging photodynamic therapy system of claim 2, wherein the flexible light guide of the therapeutic light system, and the flexible drug delivery catheter of the drug delivery module are inserted into the operative channel of the flexible transducer of the real-time imaging component.

4. The endoscopic imaging photodynamic therapy system of claim 3, wherein said insertion is via a flexible service catheter.

5. The endoscopic imaging photodynamic therapy system of claim 2, wherein:
(ii) said control handle of the endoscopic assembly is manual or computer-controlled and comprises a proximal grip and at least one service opening; or
(iii) said control handle of the endoscopic assembly has a second service opening adapted for washing the tissue, suction from the tissue, or insertion of needle biopsies to sample cells from an abnormal area for laboratory testing.

6. (canceled)

7. The endoscopic imaging photodynamic therapy system of claim 2, wherein said operation handle of the endoscopic assembly comprises means for aiming and bending the flexible transducer with the flexible drug delivery catheter and the flexible light guide towards the feto-placental unit.

8. The endoscopic imaging photodynamic therapy system of claim 1, wherein:
said real-time imaging component is ultrasound (US), magnetic resonance imaging (MRI), computerized tomography (CT), positron emission tomography (PET), light-based video, or any combination thereof; or
(ii) said real-time imaging component also measures the size of the feto-placental unit.

9. (canceled)

10. The endoscopic imaging photodynamic therapy system of claim 2, wherein said light source of said therapeutic light system is a diode laser, preferably with several variable output channels.

11. The endoscopic imaging photodynamic therapy system of claim 10, wherein said diode laser emits a light beam with a wavelength that matches one or more of the absorption peaks of the photosensitizer drug.

12. The endoscopic imaging photodynamic therapy system of claim 2, wherein said flexible light guide of said therapeutic light system is equipped with front-end optics.

13. The endoscopic imaging photodynamic therapy system of claim 2, wherein said flexible light guide of said therapeutic light system is inserted into the feto-placental unit or to its close proximity either:
simultaneously with the flexible drug delivery catheter of said drug delivery module via the operative channel of the flexible transducer; or
(ii) following the insertion of the flexible drug delivery catheter of said drug delivery module.

14. (canceled)

15. The endoscopic imaging photodynamic therapy system of claim 2, wherein said operating switch for said therapeutic light system is a pedal.

16. The endoscopic imaging photodynamic therapy system of claim 1, wherein:
the operative channel is inserted within the flexible transducer;
(ii) the operative channel is inserted in a groove of the flexible transducer;
(iii) the operative channel is attached to the flexible transducer along its full length; or
(iv) the operative channel has connecting means for attachment to a groove of the flexible transducer.

17-19. (canceled)

20. The endoscopic imaging photodynamic therapy system of claim 1, wherein the flexible transducer has connecting means for attachment to a groove of the operative channel.

21-24. (canceled)

25. A method for focused tissue ablation of a feto-placental unit in an extrauterine pregnancy (EUP) in a female patient using an endoscopic imaging photodynamic therapy system comprising an endoscopic assembly, a real-time imaging component, a drug delivery module, and a therapeutic light system, said method comprising:
inserting the endoscopic assembly into the vaginal tract of the female patient with EUP;
(ii) inserting a flexible transducer with an operative channel of the real-time imaging component through a service opening of the endoscopic assembly;
(iii) guiding the flexible transducer to the target EUP through the vaginal tract and locating the target EUP with the aid of said real-time imaging component;
(iv) inserting a flexible drug delivery catheter of the drug delivery module through the operative channel of the flexible transducer, pushing towards the target EUP, and positioning and aligning the flexible drug delivery catheter with a navigator dial or computer-operated means under real-time imaging guidance to the appropriate injection site at or close to the target EUP;
(v) injecting a photosensitizer drug directly into the target EUP with a drug delivery means of the drug delivery module;
(vi) retracting the flexible drug delivery catheter;
(vii) inserting a flexible light guide of the therapeutic light system through the operative channel of the flexible transducer, pushing towards the target EUP, and positioning the flexible light guide adjacent to the target EUP;
(viii) delivering a therapeutic light dose to said target EUP, thus inducing photodamage and tissue ablation of the feto-placental unit in the target EUP; and
(ix) retracting the endoscopic imaging photodynamic therapy system.

26. A method for focused tissue ablation of a feto-placental unit in an extrauterine pregnancy (EUP) in a female patient using an endoscopic imaging photodynamic therapy system comprising an endoscopic assembly, a real-time imaging component, a drug delivery module, and a therapeutic light system, said method comprising:
inserting the endoscopic assembly into the vaginal tract of the female patient with EUP;
(ii) inserting a flexible transducer with an operative channel of the real-time imaging component through a service opening of the endoscopic assembly;
(iii) guiding the flexible transducer to the target EUP through the vaginal tract and locating the target EUP with the aid of said real-time imaging component;
(iv) inserting a flexible drug delivery catheter of the drug delivery module and a flexible light guide of the therapeutic light system concomitantly through the operative channel of the flexible transducer, pushing towards the target EUP, and positioning and aligning the flexible drug delivery catheter with a navigator dial or computer-operated means under real-time imaging guidance to the appropriate injection site at or close to the target EUP;
(v) injecting a photosensitizer drug directly at the target EUP with a drug delivery means of the drug delivery module;
(vi) positioning the flexible light guide adjacent to the target EUP;
(vii) delivering a therapeutic light dose to said target EUP, thus inducing photodamage and tissue ablation of the feto-placental unit in the target EUP; and
(viii) retracting the endoscopic imaging photodynamic therapy system.

27. The method of claim 25, wherein the flexible drug delivery catheter of the drug delivery module and the flexible light guide of the therapeutic light system are inserted through a flexible service catheter inserted into the operative channel of the flexible transducer.

28. The method of claim 26, wherein the flexible drug delivery catheter of the drug delivery module and the flexible light guide of the therapeutic light system are inserted through a flexible service catheter inserted into the operative channel of the flexible transducer.
